(19) United States
(12) Patent Application Publication
(10) Pub. No.: US 2023/0031285 A1
(43) Pub. Date: Feb. 2, 2023

(54) System for Disinfecting a Touch Screen

(71) Applicant: Proximity Systems, Inc., Tomball, TX (US)

(72) Inventors: Jeremy Goza, Tomball, TX (US); Michael Murphy, Tomball, TX (US)

(21) Appl. No.: 17/789,373

(22) PCT Filed: Jan. 7, 2021
(86) PCT No.: PCT/US21/12474

§ 371 Date: Jun. 27, 2022

Related U.S. Application Data
(60) Provisional application No. 62/957,979, filed on Jan. 7, 2020.

(51) Int. Cl. A61L 2/24; A61L 2/10

(52) U.S. Cl. CPC A61L 2/24; A61L 2/10; A61L 2202/11; A61L 2202/14; A61L 2202/16

(57) ABSTRACT

A system mountable on a mobile device for disinfecting the mobile device the system comprising a bezel having a generally rectangular opening generally in register with the touch screen of the mobile device. Sources of UV light and reflectors project UV light onto the touch screen during a disinfection cycle. A motion sensor detects the presence of a user of the mobile device and turns the UV light source off. A timer initiates the disinfection cycle if no motion is detected for a predetermined period of time.

### CROSS REFERENCE TO RELATED APPLICATION

This application claims priority to U.S. Application No. 62/957,979 filed on Jan. 7, 2020, the disclosure of which is incorporated herein by reference for all purposes.

### FIELD OF THE INVENTION

The present invention relates to a system for disinfecting a touch screen and, more particularly, to a system for disinfecting a mobile device touch screen using ultraviolet (UV) light.

### BACKGROUND OF THE INVENTION

The prior art abounds with numerous disinfection systems which can be used with various types of UV light sources. In particular, UV disinfecting is widely used in hospitals, healthcare facilities, doctor's offices, and similar environments where germs can easily spread by virtue of human interaction with patients, instruments, surfaces, and the like. There are many commercially available UV disinfectant systems which can be installed in hospitals or similar facilities where curtailment of the spread of germs is paramount. U.S. Pat. No. 9,901,652, which is incorporated herein by reference for all purposes, discloses a UV disinfection apparatus that can be used in a hospital or similar facility.

With the advancement of technology, the use of mobile devices such as phones, tablets, and the like has become more and more common. There are various systems for disinfecting mobile devices. U.S. Pat. No. 8,964,405, which is incorporated herein by reference for all purposes, discloses a UV disinfection device for disinfecting a phone. The phone is fully enclosed within the device such that no UV light escapes. U.S. Pat. No. 9,339,573, which is incorporated herein by reference for all purposes, discloses a cabinet for disinfecting multiple tablet devices at once. Again, the devices are fully enclosed within the cabinet during disinfection.

The present invention provides a UV disinfection device for disinfecting mobile devices without the need to enclose the mobile device inside a cabinet or other enclosure.

### SUMMARY OF THE INVENTION

In one aspect, the present invention relates to a system for disinfecting a touch screen.

In another aspect, the present invention relates to a system for disinfecting a mobile device.

In yet another aspect, the present invention relates to a UV disinfection system mountable on the front of a mobile device.

These and further features and advantages of the present invention will become apparent from the following detailed description, wherein reference is made to the figures in the accompanying drawings.

### BRIEF DESCRIPTION OF THE DRAWINGS

FIG. 1 is a perspective view of one embodiment of the present invention.

FIGS. 2 and 3 are partial views depicting features of the embodiment of FIG. 1.

FIG. 4 is an exploded rear view of the embodiment of FIG. 1.

FIG. 5 is a partial view of the embodiment of FIG. 1.

FIG. 6 is a top plan view of the embodiment of FIG. 1.

FIGS. 7-10 depict the embodiment of FIG. 1 in various stages of assembly.

### DETAILED DESCRIPTION OF PREFERRED EMBODIMENTS

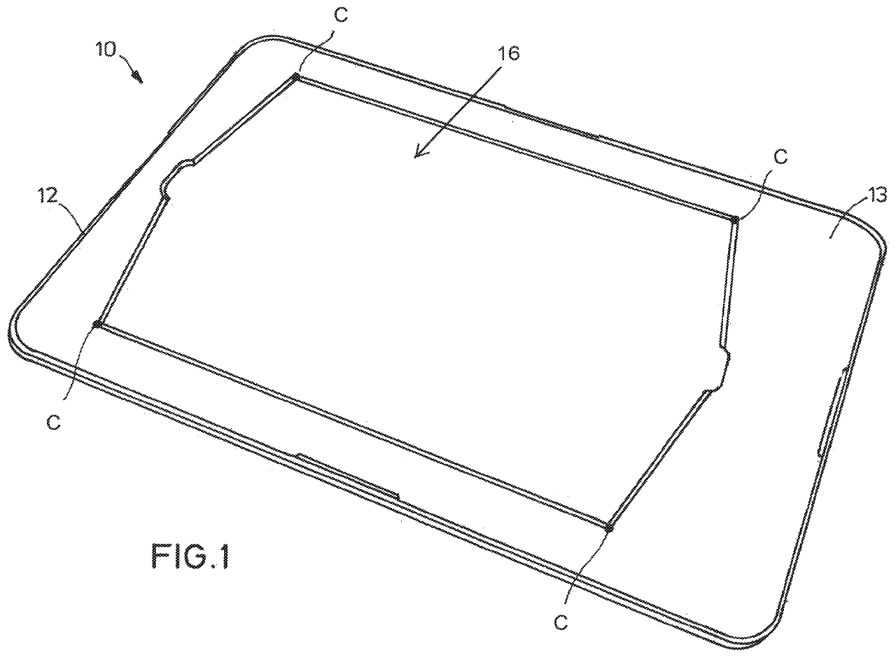

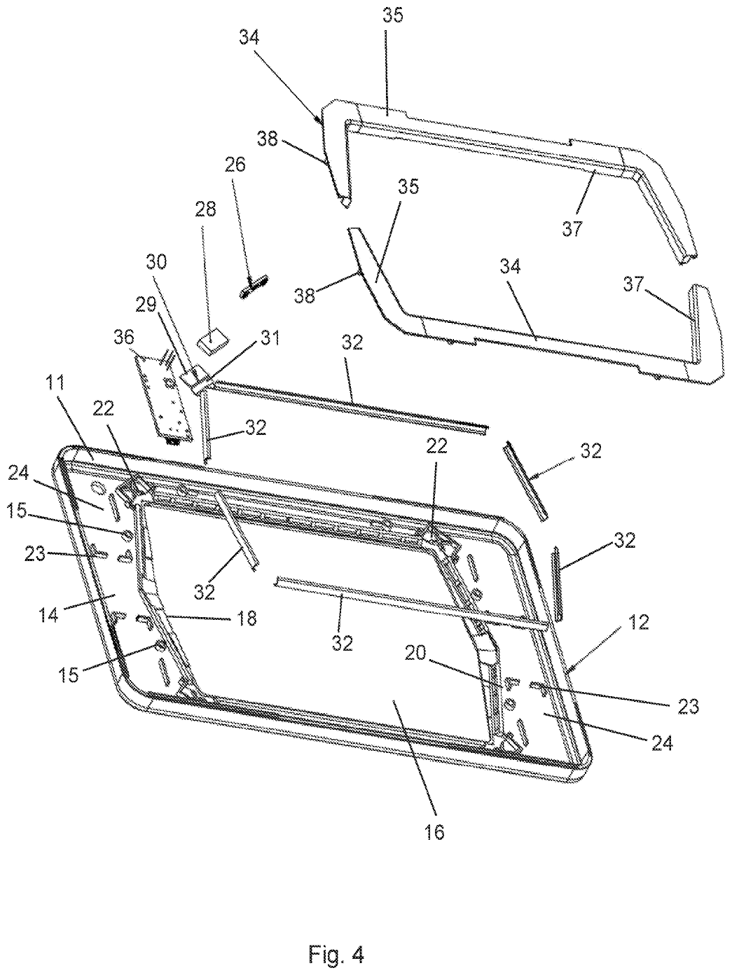

Referring first to FIGS. 1 and 4, the system of one embodiment of the present invention shown generally as 10, comprises a generally rectangular bezel 12 having a front surface 13, a back surface 14, a peripheral wall 11, and central opening 16. Bezel 12 is shaped and sized such that it can be mounted onto the front of a mobile device, including but not limited to a tablet or phone, with the touch screen of the mobile device generally aligned with opening 16. It will be appreciated that the exact size and shape of bezel 12 and of opening 16 can vary depending on the mobile device on which it will be mounted.

As depicted, the system 10 of the present invention mounts onto the front of a mobile device with peripheral wall 11 surrounding the peripheral edge of the mobile device, wall 11 snugly engaging the edge. Wall 11 can have strategically positioned openings/apertures to align with charging ports, power and volume buttons, camera lenses, and the like, of the mobile device. Such openings are not shown in the drawings and will vary depending on the specific model of the mobile device.

Another way of mounting system 10 can include the use of a plate (not shown) which fastens to the wall 11 of bezel 12, thereby at least partially encasing the mobile device between the bezel 12 and plate.

The system 10 can be retrofitted to work with existing devices. Or the system 10 may be built into a bezel of a touch screen such that it is a permanent piece of the mobile device.

The system 10 of the present invention disinfects the touch screen or surface of a device using multiple UV lights and reflectors as will be described more fully hereafter. The UV lights may be positioned in the sides and/or corners of the bezel 12. In a preferred embodiment, UV light will shine from corners C to bathe the screen of the mobile device.

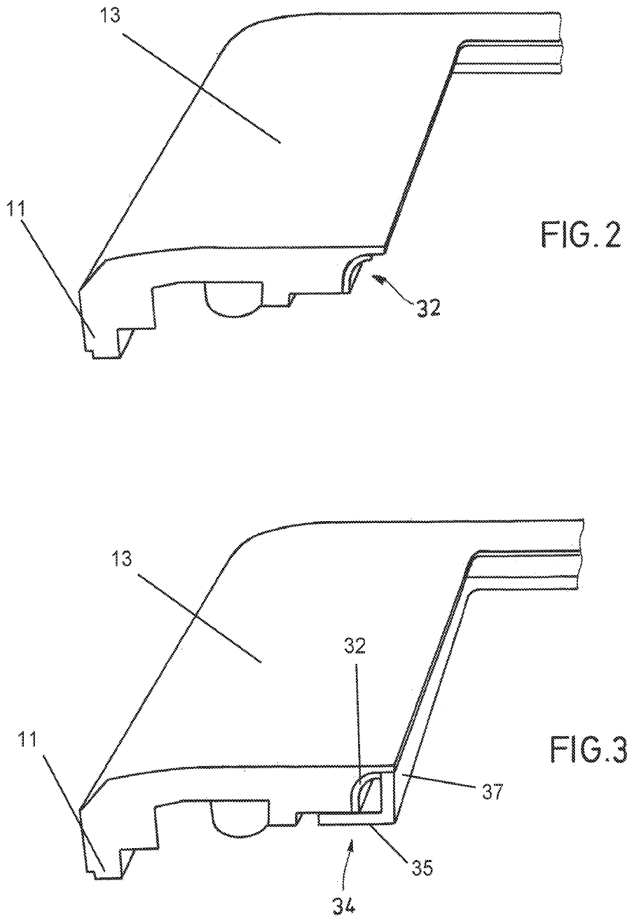

Turning now to FIGS. 2 and 3, there are shown partial views in section depicting one embodiment of the present invention. FIG. 2 shows one embodiment of a reflector 32 positioned in bezel 12. FIG. 3 shows transparent frame 34 positioned around reflector 32. Reflectors 32 and transparent frames 34 will be described more fully hereafter.

Turning then to FIG. 4, there are shown additional details of the system 10 of the present invention. FIG. 4 is an exploded view of the present invention. Surrounding opening 16 is a peripheral edge 18. A series of walls 20 which are arcuate when viewed in transverse cross-section are formed on back surface 14 around peripheral edge 18. Walls 20 form mounting grooves for reflectors 32. Positioned adjacent each corner of opening 16 is a UV light receptacle 22. Various projections 23 from back surface 14 form multiple receiving formations 24.

As seen in FIG. 4, there is a UV light source 26 to be positioned in UV light receptacle 22. The UV light source 26 can take many forms, including UV bulbs, light emitting diodes (LEDs), and the like. In a preferred embodiment, UV light source 26 is in the form of a UV LED on a printed circuit board (PCB). UV light source 26 can emit any UV wavelengths, e.g., UV-A, UV-B, or UV-C. In a preferred embodiment, UV light source 26 emits UV-C light, more particularly in wavelengths of 260-275 nm. Heat sink block 28 is positioned behind UV light source 26 within receptacle 22 and prevents overheating of UV light source 26. Heat sink block 28 and UV light source 26 are held in position by means well known to those skilled in the art. In a preferred embodiment, and as will be shown more fully hereafter, transparent frames 34 also help keep heat sink block 28 and UV light source 26 in position. It will be appreciated that heat sink block 28 may not be required depending on the type of UV light source used. Corner reflector assembly 30 is also mounted in receptacle 22. Corner reflector assembly 30 is comprised of plate 29 and reflector 31. Plate 29 is mounted in receptacle 22, preferably between heat sink 28 and back surface 14. Corner reflector assembly 30 is positioned such that reflector 31 extends out proximate UV light source 26 and reflects UV light from UV light source 26 toward the mobile device touch screen and toward elongate reflectors 32. In a preferred embodiment, reflector 31 is arcuate. It will be understood that while only a single UV light source 26, heat sink block 28, and corner reflector assembly 30 are depicted in FIG. 4, it is preferred that each UV light receptacle 22 has the same configuration of components. Thus, UV light sources will be positioned at each corner of bezel 12. It will further be appreciated that the UV light source and reflectors may be positioned along the sides of the houses instead of or in addition to the corners. The exact number and position of UV light sources and reflectors can vary depending upon the size of the touch screen. For example, one embodiment of the present invention may have multiple UV light sources positioned at each corner, while another embodiment of the present invention may only use UV light sources positioned at two diametrically opposite corners.

Returning to FIG. 4, an array of elongate reflectors 32 are disposed along peripheral edge 18 and held in place against inner walls 20 by means well known to those skilled in the art, for example, by snapping them into place or through the use of adhesive. Elongate reflectors 32 are positioned such that the light from UV light source 26 is reflected outwardly to bathe the entire touch screen of the mobile device in UV light without reflecting UV light toward the eyes of a user. Elongate reflectors 32 are preferably arcuate in shape when viewed in transverse cross-section. It will be understood that the exact curvature, angle, or length of elongate reflectors 32 can vary, as long as they direct the UV light to bathe the touch screen/surface of the device. As shown in FIG. 4, not all reflectors 32 are the same length.

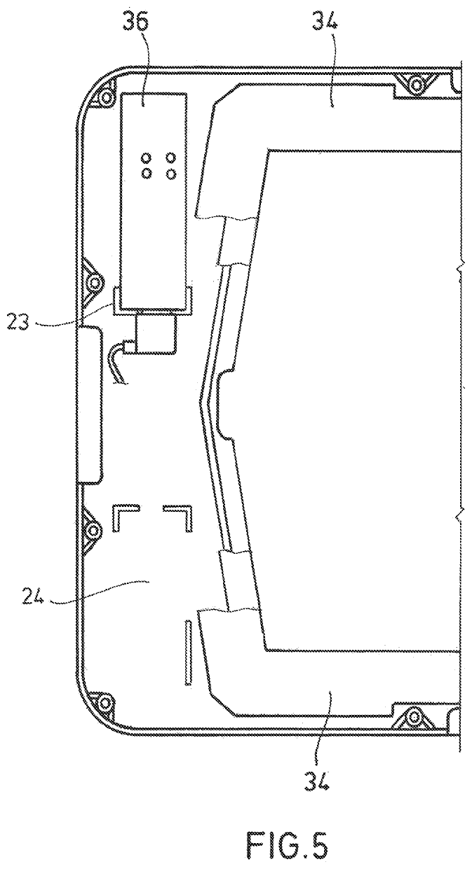

In a preferred embodiment, transparent frames 34 are mounted over the corner assemblies and elongate reflectors 32. As depicted transparent frames 34 are generally L-shaped when viewed in transverse cross-section, having a first section 35 generally parallel to surface 14, and a second section 37 generally perpendicular to surface 14. As shown in FIG. 4, there are two transparent frames 34, but the exact number and shape of them can vary. Transparent frames 34 will be made of a UV transparent material which allows at least UV light to pass through, e.g., fluorinated ethylene propylene. As best seen in FIGS. 4 and 5, transparent frames 34 have projections 38 which fit into receptacles 15 on back surface 14. Transparent frames 34 can be attached by any means wells known to those skilled in the art, for example, by snapping them in place or affixing with screws. Transparent frames 34 protect the components of the present invention from dust, particles, or the like without impeding the transmission of UV light across the touch screen of the mobile device.

At least one PCB driver/controller 36 is mounted in a receiving formation 24 to control the operation of the UV light source 26.

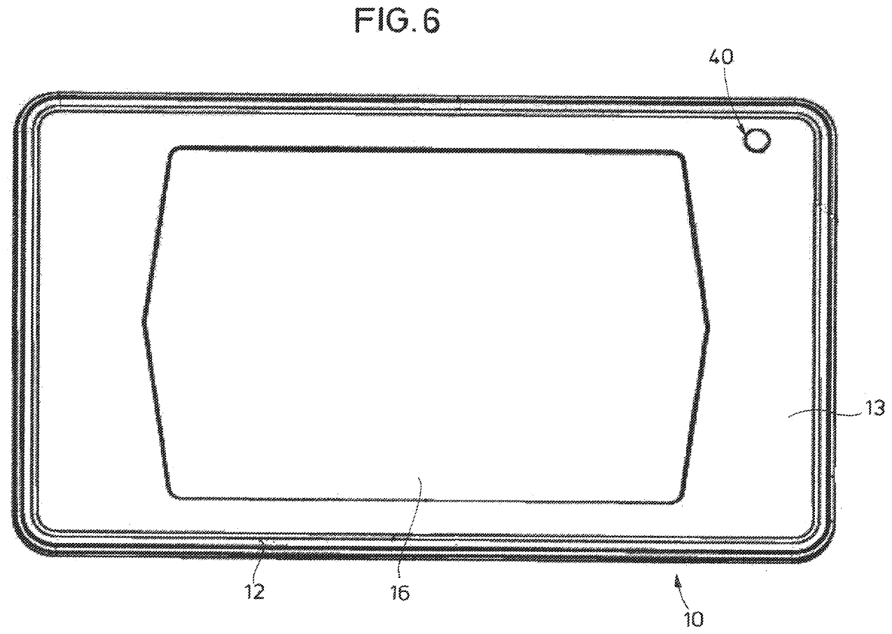

In a preferred embodiment, the system 10 of the present invention includes a motion sensor 40 (see FIG. 6) and a timer (not shown), both controlled by driver/controller 36. The motion sensor 40 detects motion, e.g., the presence of a user, and controls the UV light source 26 in response thereto. The motion sensor can be a passive infrared sensor, capacitive sensor, inductive sensor, infrared sensor, heat or thermo sensor, imager, or the like. Motion sensor 40 monitors the area around at least the touchscreen and thus detects the motion from a person using the mobile device.

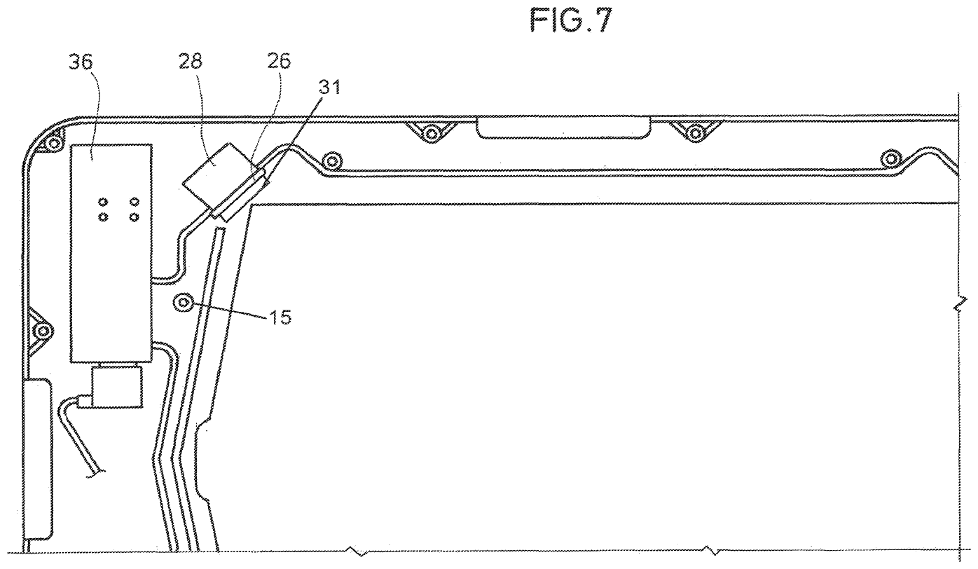

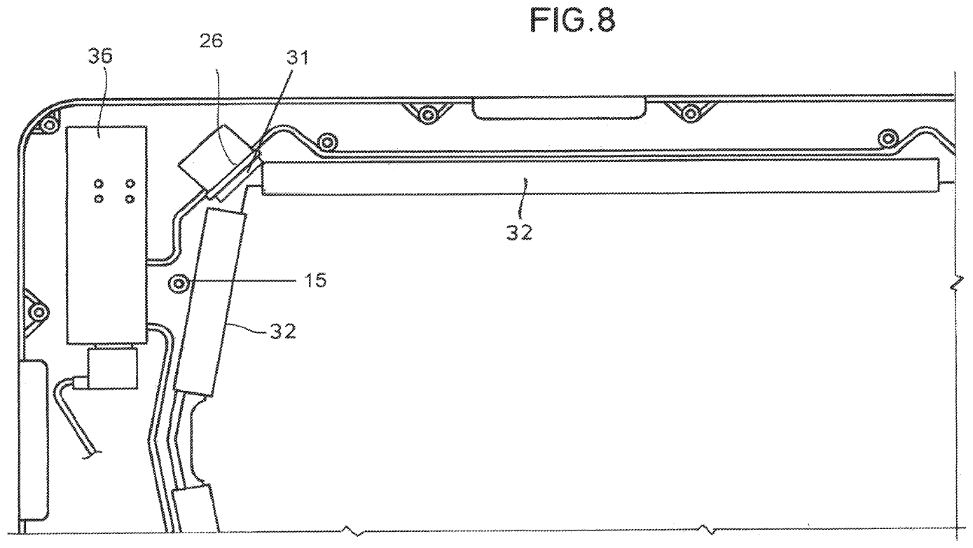

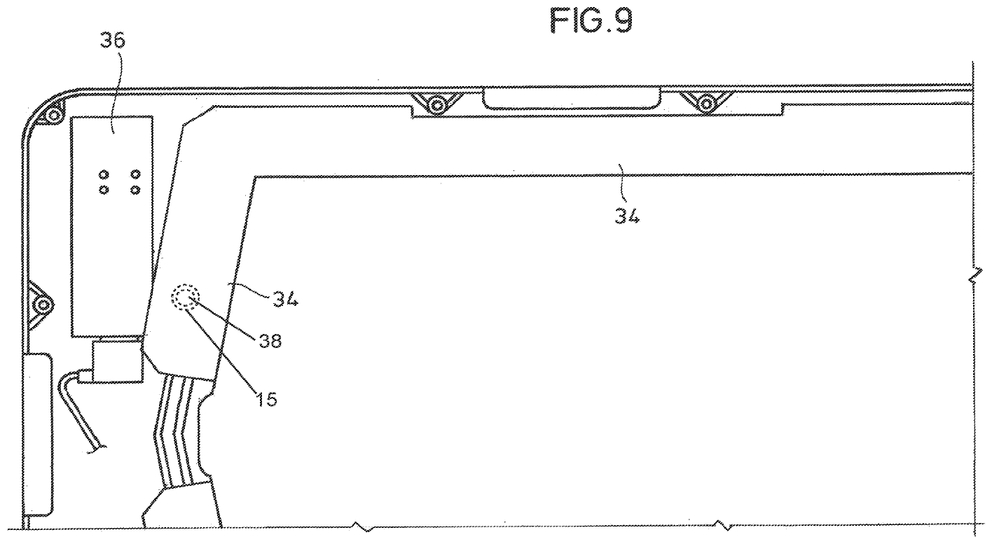

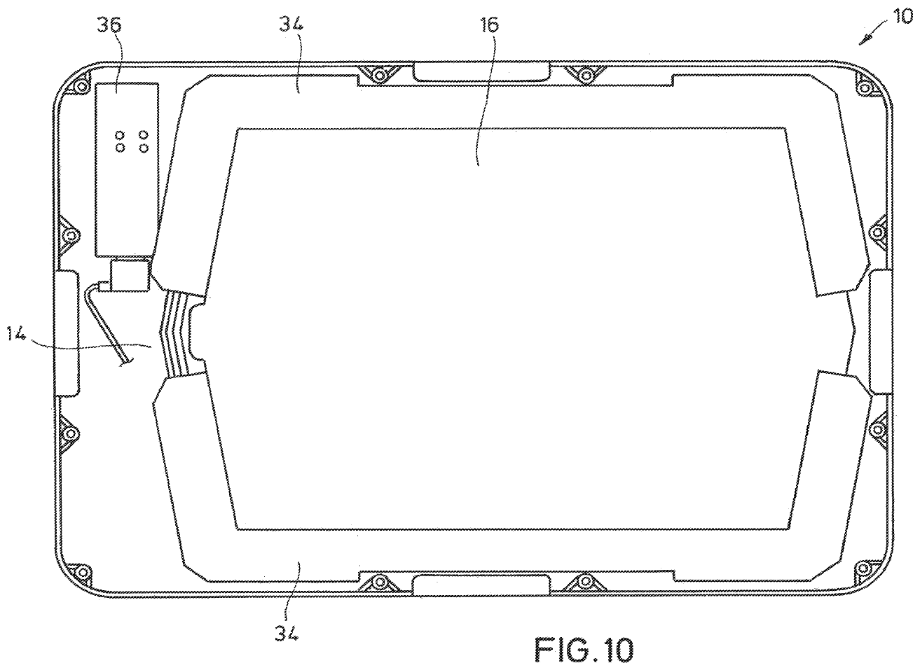

FIGS. 7-10 provide closer views of the features of the present invention as they are assembled. FIG. 7 shows the driver/controller 36, UV light source 26, and heat sink block 28 mounted in bezel 12. FIG. 8 shows elongate reflectors 32 positioned around opening 16. FIG. 9 shows transparent frames 34 positioned over UV light receptacle 22 and elongate reflectors 32. FIG. 10 shows the system 10 fully assembled.

It will be understood that the system includes necessary connections, power sources and the like which are not expressly set forth herein but which are well known to those of ordinary skill in the art.

In operation, when motion sensor 40 detects motion, e.g., the presence of a user, the UV light source 26 is turned off and remains off for the duration of the motion. When the detected motion ceases, a timer begins a delay period, e.g., 60 seconds, before initiating a disinfection cycle. This ensures that the disinfection cycle does not begin when a user is still nearby but not moving, e.g., while the user is reading the screen. If motion is detected during the delay period, the delay period is stopped. When the motion ceases, the delay period restarts from the beginning. If no motion is detected during the delay period, the UV light source 26 is turned on for a disinfection cycle of a predetermined length of time, e.g., 5 minutes. After the disinfection cycle is complete, the UV light source 26 is turned off again and remains off until motion is detected again. If motion is detected before the disinfection cycle ends, the UV light source is turned off again and when the motion ceases, the delay period begins again.

If desired, the system 10 of the present invention can be programmed to periodically initiate a disinfection cycle even without detected motion. For example, if no motion has been detected for 2 hours, a disinfection cycle is initiated. Thus, the mobile device remains disinfected from other airborne bacteria, viruses, and the like.

The system 10 of the present invention provides many advantages over the prior art. Some prior art systems require the item to be disinfected to be inside a specialized disinfection enclosure. The disinfection system of the present invention is incorporated into a case or housing which fits on the mobile device itself. Thus, the user is able to access the mobile device as needed. This is also particularly useful for travel. The motion sensor and timer ensure that the user is not inadvertently exposed to UV light.

It will also be appreciated that while the system 10 of the present invention has been discussed with respect to mobile devices, it may also be placed on touch screens which are not part of mobile devices, i.e., kiosk screens. The bezel 12 could be affixed to a kiosk screen by means well understood to those skilled in the art.

Although specific embodiments of the invention have been described herein in some detail, this has been done solely for the purposes of explaining the various aspects of the invention, and is not intended to limit the scope of the invention as defined in the claims which follow. Those skilled in the art will understand that the embodiment shown and described is exemplary, and various other substitutions, alterations and modifications, including but not limited to those design alternatives specifically discussed herein, may be made in the practice of the invention without departing from its scope.

1. A system for disinfecting a mobile device having a touch screen, the system comprising:
a bezel positionable on said mobile device, said bezel having a first surface, a second surface, a peripheral wall, and an opening;
at least one UV light source mounted on said second surface proximal said opening;
at least one reflector mounted on said second surface proximal said at least one UV light source and said opening to direct light from said UV light source toward said mobile device; and
a controller mounted on said second surface and operative to control said UV light source.

2. The system of claim 1 further comprising:
a motion sensor operative to monitor an area encompassing at least said mobile device and detect motion in said area;
a timer;
said controller being operatively connected to said motion sensor and timer and configured to turn said UV light source off when motion is detected, and to turn said UV light source back on when no motion is detected for a predetermined period of time.

3. The system of claim 2, wherein said motion sensor is one of passive infrared sensors, capacitive sensors, inductive sensors, infrared sensors, heat or thermo sensors, and imagers.

4. The system of claim 1, further comprising at least one transparent frame mounted on said second surface, said transparent frame being between said at least one reflector and said opening.

5. The system of claim 1, wherein said UV light source comprises UV LEDs.

6. The system of claim 1, wherein said UV light source emits UV-C light.

7. The system of claim 1, wherein a plurality of UV light sources are mounted on said second surface and operative to shine UV light on said mobile device.

8. The system of claim 1, further comprising a plate attachable to the back of said bezel to encase said mobile device between said bezel and said plate.
